(19) United States
(12) Patent Application Publication
(10) Pub. No.: US 2023/0248760 A1
(43) Pub. Date: Aug. 10, 2023

(54) THERAPEUTIC COMPOSITION FOR ARRESTING, PREVENTING AND REVERSING DENTAL DISEASE

(71) Applicant: Mohamed HARUNANI, Rockford, IL (US)

(72) Inventor: Mohamed HARUNANI, Rockford, IL (US)

(21) Appl. No.: 18/301,186

(22) Filed: Apr. 14, 2023

Related U.S. Application Data
(63) Continuation-in-part of application No. 17/190,532, filed on Mar. 3, 2021.
(60) Provisional application No. 63/091,310, filed on Oct. 14, 2020.

(51) Int. Cl. A61K 33/10; A61K 33/42; A61K 31/522; A61K 45/06; A23L 27/30; A61K 9/00; A61K 35/747

(52) U.S. Cl. CPC A61K 33/10; A61K 33/42; A61K 31/522; A61K 45/06; A23L 27/34; A61K 9/0056; A61K 35/747

(57) ABSTRACT

The present invention generally relates to a therapeutic composition for arresting, preventing and reversing dental disease. More specifically, the present invention relates to a alkaline based composition and method of delivery for improving oral health and delivering nutrients, agents, or combinations thereof.

### CROSS REFERENCE PRIORITY CLAIM AND RELATED APPLICATIONS

This application is a continuation-in-part of co-pending U.S. patent application Ser. No. 17/190,532, filed Mar. 3, 2021, which claims the benefit of priority to U.S. Provisional Application No. 63/091,310, filed Oct. 14, 2020. The disclosures set forth in the referenced applications are incorporated herein by reference in their entireties.

### BACKGROUND

The present invention generally relates to a therapeutic composition for arresting, preventing and reversing dental disease. More specifically, the present invention relates to an alkaline-containing composition and method of delivery for improving oral health and delivering nutrients, agents, or combinations thereof.

Oral hygiene is one of the most important aspects of personal care among consumers. Consumers worldwide use different types of products for oral care as a part of maintaining dental hygiene. Not all oral disease arises from poor oral hygiene. Medications, head or neck radiation treatment, and Sjogren's syndrome for example are significant causes or oral diseases. Poor oral hygiene, increased use of citrus or other acid containing ingredients in food, and increased sugar consumption keep the oral cavity's pH low (acidic) which increases demineralization or tooth decay. Decay, gum disease, and discolored teeth are all symptoms of poor oral health. Generally, teeth compose a hard enamel outer layer and a softer inner dentin layer that cover the pulp (composed of a complex network of nerves, blood vessels, and lymphatics). The layers are arranged in a crystalline structure called hydroxyapatite composed of calcium and phosphate ions and contain trace minerals such as strontium, magnesium, lead, and fluoride. The enamel is formed in rods within a predominantly inorganic matrix material containing hydroxyapatite, protein, and other compounds. Dentin has an increased type I collagen content and decreased mineral content when compared to enamel. Dentin contains fluid-filled channels (tubules) and decays more rapidly than enamel.

A succession of oral bacteria produces decay by forming plaque deposits on the teeth. For example, the bacterium Streptococcus mutans (S mutans) produces glucan that helps bacteria adhere to teeth and form plaque deposits. Initial colonization by S mutans is followed by a succession of bacteria that produce increasing amounts of lactic acid causing the removal of minerals (demineralization) from the tooth and consequently weakening it. Plaque bacteria metabolize sugars (fructose, sucrose, and glucose) and carbohydrates, resulting in the formation and secretion of lactic acid as a byproduct of their metabolization. At 5.5 pH, demineralization (also called cavitation or decay) begins and continues through the enamel until dentin is penetrated. Since dentin dissolves at a higher pH of 6.2-6.5 (less acidic environment), the decay process speeds up as decay proceeds. Tooth enamel is soluble in acids. During the demineralization process, minerals (calcium ions and phosphate ions) are removed from the enamel and dentin until the enamel collapses. In addition, the defect created cannot be replaced by remineralization processes. Cycles of remineralization and demineralization continue throughout the decay process with demineralization created by the ingestion of acidic food/drinks and by acid forming bacteria resulting in an acidic oral environment while remineralization (replacement of the removed mineral from saliva) occurring during periods of neutral or alkaline pH. Enamel may be remineralized between meals by replacing missing calcium and phosphate ions during periods of alkaline or neutral pH (7 or higher) by ions present in the saliva, assuming that there is adequate calcium and phosphate available in a bio-available form. The presence of fluoride hastens this process. Saliva is necessary for repairing tooth structure and inhibiting tooth decay. Saliva production is essential to several biological functions including swallowing, digestion, and lubrication of the oral cavity. Salivary glands may become impaired and/or damaged due to accident, trauma, illness, disease, and/or medical treatments, including surgery, medications, radiation therapy, and chemotherapy treatments. Decreased saliva is the largest cause of oral issues in the elderly; it causes tooth decay, tooth loss, dental abscesses, and mucositis. Other significant health conditions, including malnutrition, pain, and/or infections, can also be related to a decreased salivary flow and/or altered oral environment.

Further, a common scenario and cause of increased tooth decay involves the usage of bottled water by children and adults, which most often is not fluoridated. This results in two consequences, which include the unavailability of fluoride in the teeth of the growing children, and the lack of fluoride does not allow for the strengthening of the surface layer, and so these teeth are more prone to breakdown. Fluoride converts as the hydroxyapatite crystal to fluorohydroxyapatite crystal, which is much less soluble. Fluorapatite (FA) is formed when fluoride exchanges with the hydroxyl groups of present hydroxyapatite crystals in the tooth, which is less soluble in acid.

Further, in nursing homes and homes for the disabled, the most common foods are soft and sticky to the teeth. As known, any carbohydrates and/or sugars are converted to lactic acid by the cariogenic bacteria. Since these teeth do not get brushed regularly or immediately, they tend to decay. In these populations, manual dexterity is also a problem, making brushing more difficult. Further, average life of restorations (fillings) is less than ten years and much shorter in these cases; due to repeatedly being redone, the extent of tooth loss is massive and at a considerable cost in terms of money, time, pain and suffering. In young patients, consumption of sugars and carbohydrates is much higher. Restorations may need to be replaced multiple times at a considerable cost in terms of money, time, pain and suffering.

In a study of 380 beverages purchased from stores in Birmingham, Ala., categorized (e.g. juices, sodas) and assessed for pH, It was found that the majority (93%, 355/380) of beverages had a pH below 4.0 and 7% (25/380) had a pH>4. Relative beverage erosivity zones based on previous studies of apatite solubility in acid indicated: 39% (150/380) of the beverages were considered extremely erosive (pH<3.0); 54% (205/380) were considered erosive (pH 3.0 to 3.99); and 7% (25/380) were considered minimally erosive (pH>4.0). These drinks usually contain citric acid, which is one of the worst of edible acids due to its pKa.

Dental erosion from beverages is primarily caused by either phosphoric acid and/or citric acid, and both are triprotic acids with 3 available [H+] enabling proton-promoted dissolution. Chelation or ligand-promoted dissolution by anionic citrate contributes to enamel demineralization by the removal of Ca²⁺ at a higher pH range approaching 6. At the erosive pH, only 3% of citrate ions are appropriately ionized to chelate Ca²⁺, indicating their contribution to the erosive process at this pH is minimal. However, if anionic citrate remains within the oral cavity for extended time intervals allowing the pH to rise to 6, chelation could play a contributing role in the erosive process. The eating of citrus fruits more than twice a day has been associated with dental erosion. Nevertheless, high concentrations of [H⁺] reflected by low pH from citric and/or phosphoric acid result in undersaturation for fluor- and hydroxyapatite leading to dental erosion. Hence, pH is the controlling parameter in determining the erosive potential of beverages.

Further, the present society consumes numerous meals on the run and fast food or in their private vehicles or public transportation with no way of cleaning or de-acidifying their oral cavity to stop the acid demineralization, whether it is from the acidic food/drink or the lactic acid being produced by the cariogenic bacteria. Additionally, most medications cause dry oral cavity and a reduction in the salivary flow, which lets the acid stay in the oral cavity longer and having a longer time to dimineralize.

Accordingly, there is a need for a composition that could improve oral health by neutralizing the oral pH, repairing damaged tooth structure, strengthening the enamel, and delivering nutrients, therapeutic agents, or combinations to improve health and well -being. Further, there is a need for a composition in a form candy that would consistently deliver ingredients that will immediately reduce acidity, encourages salivary flow and natural sucking and other movements that occur, and causes the cleansing of the oral cavity and thus enable reduction of the pathogenic load while encouraging remineralization due to providing bio-available calcium and phosphate ions over an extended period which will increase the needed contact time for remineralization. Further, there is a need for a low cost, composition in the form of candy that would naturally and permanently reverse incipient lesions without the need for a dental restoration. Further, there is a need for a composition and method of delivery that would quickly reduce acidity and encourages remineralization.

### SUMMARY OF THE CLAIMS

In some embodiments a method of improving oral health is hereby disclosed, the method comprises the steps of (1) neutralizing the oral cavity pH by administering a calcium-containing compound; (2) maintaining a steady state of neutral or alkaline pH in the oral cavity, (3) simultaneously with step (2) remineralizing tooth enamel, (4) reducing the oral bacterial load through increased salivation, oral movement and texture on the candy, and (5) delivering bioavailable calcium and phosphate ions needed over an extended period.

The method further comprises administering a delivery vehicle containing the composition that consists of at least one remineralizing agent. The remineralizing agent consists of at least one fluoride-containing compound. The delivery vehicle may also consist of at least one calcium-containing compound, such as calcium carbonate; at least one sweetener; at least one additive comprising: at least one tooth whitening agent; at least one probiotic; at least one hydrating agent; at least one energy supplement, at least one treatment agent for halitosis, and at least one germicidal agent.

The at least one calcium-containing compound may comprise tricalcium phosphate or calcium carbonate. The at least one sweetener may be selected from the group consisting of a high-intensity sweetener in a range of about 0.001 g to about 0.006 g, and an alcohol-containing like erythritol in the range of about 1 g to about 6 g which will further decrease the oral bio-load of Strep mutans without the gastric symptoms noted with use of Xylitol. The calcium carbonate may buffer a consumer's oral cavity to a pH of about 7 or greater. In some embodiments, the calcium carbonate may buffer a consumer's oral cavity to a pH of about 6.9 or greater

In some embodiments, the therapeutic composition for improving oral health comprises at least one remineralizing agent that includes at least one fluoride-containing compound; at least one calcium-containing compound; at least one sweetener; an additive, selected from one or more of the following: at least one tooth whitening agent; at least one probiotic; at least one energy supplement; at least one hydrating agent; and at least one treatment agent for halitosis, at least one germicidal agent.

### BRIEF DESCRIPTION OF THE DRAWINGS

FIG. 1 exemplarily illustrates an embodiment of a alkaline based composition and the composition is in the form of hard or soft candy or lollipop (100),

FIG. 2 exemplarily illustrates an embodiment of an end-user (102) having improved oral health on using the alkaline based composition, and

### DETAILED DESCRIPTION

The description that follows describes, illustrates and exemplifies one or more embodiments of the present invention in accordance with its principles. This description is not provided to limit the invention to the embodiments described herein, but rather to explain and teach the principles of the invention in order to enable one of ordinary skill in the art to understand these principles and, with that understanding, be able to apply them to practice not only the embodiments described herein, but also other embodiments that may come to mind in accordance with these principles. The scope of the present invention is intended to cover all such embodiments that may fall within the scope of the appended claims, either literally or under the doctrine of equivalents.

The present invention discloses an alkaline based composition for improving oral health and delivering nutrients, therapeutic agents, or combinations thereof. The composition, immediately upon use, lowers the acidity in the oral cavity to create an environment that arrests or reverses disease processes (i.e., dental caries and periodontal disease) while bringing other benefits to the consumer including, but not limited to, stronger bones and whiter, healthier teeth. The composition is in the form of a non-sugar (sucrose, fructose, or glucose) containing candy with the capacity to deliver various ingredients in a pH-controlled manner, to quickly decrease the acidity of the oral cavity and deliver the ingredients needed to prevent and/or repair teeth and also provide other therapeutic benefits in a beneficial and/or less destructive manner. Additionally, the candy has β-tricalcium phosphate (β-TCP) incorporated into it and as such, as the candy is enjoyed, the structure of the β-TCP, cleanses the tongue which causes the dissolution of the encapsulation which releases the Bio-available calcium and phosphate over an extended period.

In one embodiment, the composition comprises at least one calcium-containing compound, at least one carbonate compound, at least one active ingredient, at least one sweetener, at least one hydrating agent, at least one electrolyte, at least one ingredient that can provide bio-available calcium and phosphate ions, and at least one additive. The composition further comprises water. In one embodiment, the additive is selected from the group consisting of an antimicrobial agent, an antibacterial agent, an antiviral agent, a tooth whitening agent, a probiotic, an energy supplement, a colorant, caffeine, and a flavorant. In another mixture of encapsulated components, the composition includes at least one of vitamin, mineral, or electrolyte. In one embodiment, the additive is an agent that helps with halitosis/mal-odor (a lactobacillus or other).

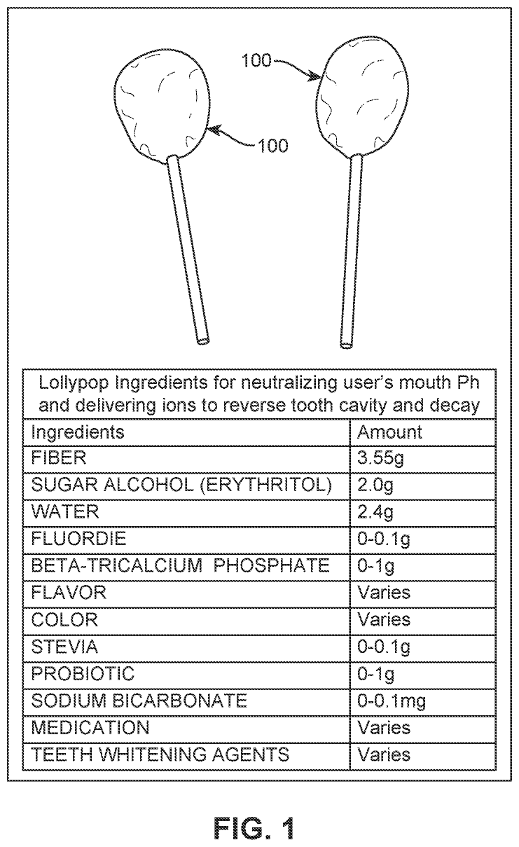

Referring first to FIG. 1, the composition is shown in the form of a lollipop 100. The composition includes at least one high-intensity sweetener component in a range of about 0.001 g to about 0.006 g. In yet another embodiment, the composition comprises fiber of about 2-6 g, and β-tricalcium phosphate (β-TCP) in a range of about 0.5 g to about 5 g, and approximately 1-6 g of a sweetener like an erythritol. In yet another composition, the mixture may include a fluoride-containing compound ranging from 0 g to about 0.2 g, about 0-5 g water, carbonate ranging from about 0.02 mg to about 20 mg, and the amount of probiotic and other therapeutic compounds will vary. In other embodiments, the candy composition can be made in a form of a hard candy, a chewy candy, a gummy candy, a jelly candy, or a combination thereof.

In one embodiment, the candy is a lollipop comprising about 4.46 g fiber like nutriose, about 2.52 g sugar alcohol, about 1.05 g water, about 0.725 g of a fluoride-containing compound, about 0.75 g β-tri-calcium phosphate, about 0.038 g high-intensity sweetener compound like Stevia, about 1 mg sodium carbonate, about 0.45 g of flavorant, and about 0.45 g of colorant.

Active ingredients in each embodiment will vary based on therapeutic needs. For example, the fluoride-containing compound forms harder enamel and inhibits decay; the tooth whitening agents contain an ingredient which will whiten teeth; the probiotics inhibit bacteria causing gum disease; the antiviral and/or antibacterial agents reduce the pathogenicity of the oral cavity, and the vitamins and minerals (the building blocks of the tooth) strengthen teeth and improve oral and general health.

Today, electrolytes are generally consumed from sports drinks, which are very acidic. This is a great way to intake electrolytes without destructive sugar and/or acid. Similarly, caffeine and other energy-boosting ingredients can be provided without the sugars and acids that increase tooth decay. The addition of carbonate can further help buffer against the acids created by consuming acids and/or plaque containing bacteria. In addition, the candy composition aids therapeutic agent delivery alongside electrolytes to help decrease their deleterious impact on teeth and to protect them from the destruction currently being experienced through the current delivery methods.

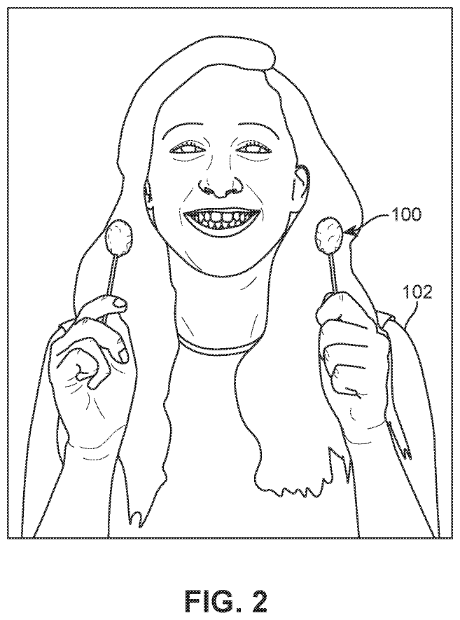

FIG. 2 exemplarily illustrates an end-user (102) with improved oral health using the alkaline based composition in the form of a lollipop 100, according to an embodiment of the present invention. The candy dissolves in the oral cavity quickly decreases oral acidity and releases beneficial ingredients. Thereby, the composition creates a favorable oral environment for oral health including remineralization of the tooth, leading to the protection of teeth and possible reversal of cavities and other diseases while also being able to deliver other beneficial ingredients.

In one formulation, the dosage of the therapeutic agents/ingredients of the composition may vary based on desired outcomes, timed release of therapeutic agents, and tailored for subjects of different ages. In different formulations, the candy may be supplied in different flavors and consistencies to appeal to different tastes and unique needs. In addition, the composition is safe for children and diabetics without gastric distress created by alcohol sugars and/or side effects of other sweeteners. Furthermore, it is safe for pets (unlike xylitol containing sweeteners).

In one embodiment, the composition of the present invention may vary depending on the condition being addressed. In another embodiment, the ingredients of the composition are microencapsulated and/or directly mixed into the candy. As the candy is consumed, the therapeutic agent is delivered in a controlled fashion without the harmful effects of sugar and acid commonly associated with candy.

The composition is configured to alter the oral environment by adding elements including, but not limited to, calcium, phosphate, and buffering ions. The composition is configured to deliver ions required for oral health simultaneously while being separated during delivery utilizing microencapsulation. The composition is configured to decrease acidity, increase salivary flow, and offer a tasty and effective means to significantly change the oral environment to quickly stop the demineralization and enhance remineralization.

The composition is configured to quickly reduce the acidity of the oral environment while delivering agents that promote oral and general health. The alkaline based composition acts as a vehicle to deliver different therapeutic agents using a candy delivery system that would quickly decrease the acidity of the oral environment, increase saliva flow, and improve oral and general health without the adverse effects associated with sugar containing candy in the oral environment.

The alkaline-based composition is configured to reduce the acidity of the oral environment quickly. In addition, the composition is configured to provide a sustained release of therapeutic calcium, phosphate, and fluoride ions into the oral cavity for the highest therapeutic impact to reduce or eradicate cariogenic challenge in plaque following sucrose intake. In addition, the composition comprises erythritol and other similar sweeteners that have proven to reduce bacteria and plaque in the oral cavity. The released calcium, phosphate, and fluoride ions can diffuse into partially demineralized tooth enamel or dentin, leading to remineralization and repair of the caries lesion. These ions can also precipitate in dentinal tubules and relieve sensitive teeth. The alkaline-based composition as a delivery system aids compliance and pleasure while improving health and reducing costs to the user, the payor, and the government by reducing the cost of treatment, thus creating significant savings for all in terms of health and wealth while improving wellbeing. This composition is ideal for use after a meal when one cannot brush their teeth, for snacking, and the perfect solution to reduce decay in elderly populations, particularly in a nursing home environment. The life expectancy of dental restoration is about ten years. Thus, patients may need treatment multiple times, but a tooth that has been remineralized can last a lifetime without the need for continued treatment.

In an embodiment, the present composition comprises a three part system. The three part system may take the form of a delivery vehicle for delivery of the composition into the oral cavity over a period of time. This period of time is known as the “residence time” of the composition. Increasing residence time increases remineralization of tooth enamel because it increase the amount of time the calcium-, phosphate-, and fluoride-containing compounds are in constant contact with the tooth enamel. The amount of time the compounds are in contact with the tooth enamel is known as the “contact time.”

The first part of the three-part treatment system stops the acidification process in the oral cavity through the use of a calcium-containing compound. In some embodiments, this is calcium carbonate, however a person having ordinary skill in the art would know any suitable calcium-containing compound would be sufficient. In some embodiments, the calcium-containing compound is β-tri-calcium phosphate (“tricalcium phosphate” or “TCP”). The calcium-containing compound present in the composition reacts with saliva in the oral cavity to form a buffer solution in sufficient quantity to stop acidification of the tooth enamel.

The second step involves maintaining a neutral or basic pH, i.e. greater than about 6.9 pH, in the oral cavity. Maintaining a neutral or basic pH in the oral cavity allows the composition administered via the delivery vehicle to remineralize teeth in the oral cavity in step three; as such, maintaining a neutral or basic pH in some embodiments must occur concurrently or simultaneously with the third step, remineralization. Maintaining a neutral or basic pH in the oral cavity is achieved via the use of a calcium-containing compound, similar to the first step. However, in some embodiments, the calcium-containing compound must be maintained in stoichiometric excess with reactants in the oral cavity, such as saliva and saliva's acid containing components. This results in a buffer system for the duration that the user consumes the composition. If the calcium-containing compound is not maintained in stoichiometric excess, buffering will cease and the oral cavity will no longer be at a neutral or basic pH and prevent remineralization.

In some embodiments, the third step, which may occur simultaneously with the second step and only after the first step (the stopping of acidification), is the remineralization of tooth enamel. This happens by virtue of the calcium-, phosphate-, and fluoride-containing compounds remineralizing the tooth enamel. The fluoride-containing compound may remineralize the tooth enamel when the buffer solution is formed in step two and the oral cavity is maintained at a steady state pH of about 6.9 or greater while these ions are in a bio-available form. In some embodiments, these compounds remineralize teeth when the buffer solution is formed in step two and the oral cavity is maintained at a steady state pH of about 6.9 or greater.

Additionally, in some embodiments, the effectiveness as a hard candy or lollipop 100 has benefits compared to other forms due to the increased time it takes to consume it, and thus, increased the residence time in the oral cavity of the hard candy or lollipop 100. The hard candy, which in some embodiments is in the form of a lollipop 100 or similar, has a longer residence time in the oral cavity. Residence time directly affects how much remineralization occurs. Longer residence times in the oral cavity may increase remineralization. In some embodiments, the composition may allow for buffer solutions that can last the duration of the residence time of the hard candy by maintaining the calcium-containing compound in stoichiometric excess when compared to the other chemicals present in the composition and reactants present in the oral cavity.

In some embodiments, the composition is in the form of a water-soluble powder, and the delivery vehicle is the water-soluble powder mixed in a solution primarily consisting of water. The water-soluble powder, in some embodiments, has the benefit that it can be mixed with any drink, however it has a lower residence time in the oral cavity compared to the hard candy or lollipop 100 form. To increase the residence time, the water-soluble powder suspended in a drink could be sipped over a longer comparative period of time.

In other or the same embodiments, residence time is directly connected to contact time. Contact time, in some embodiments, refers to the amount of time the fluoride-containing compound is in contact with the tooth enamel. Increasing residence time of the composition via the delivery vehicle increases the contact time of the fluoride-based compound with the tooth enamel. Residence time is increased by increasing the amount of the calcium-containing compound such that the buffering process is prolonged. Contact time is increased by virtue of increasing residence time and increasing the amount of fluoride-containing compounds. The contact time can range from 5 to 15 minutes in some embodiments. More specifically, the contact time can range from 7 to 13 minutes in some embodiments. More specifically, the contact time can range from 8 to 12 minutes in some embodiments. Longer contact times in the oral cavity may further increase the amount of remineralization that occurs by virtue of increased residence time of the composition via the delivery vehicle.

By virtue of increased residence time of the composition via the delivery vehicle and increased contact time of the calcium-, phosphate-, and fluoride-based compounds, the hard candy or lollipop 100 form of the composition exhibits a timed therapeutic release. By exhibiting a timed release, the hard candy or lollipop 100 may increase the therapeutic effects of the fluoride-based compound and consequently may increase remineralization of the teeth over a period of time.

In some embodiments, the hard candy or lollipop 100 form of the composition further comprises a textured surface. The textured surface consists of small ridges or bumps. These small ridges or bumps are included by virtue of the TCP containing compound's properties. TCP containing compounds are encapsulates, and are generally in the form of a granular powder, and the powder's texture imparts the composition with a textured surface. The textured surface aids in abrasive cleaning of the tongue, gums, and soft surfaces of the oral cavity. The textured surface further aids in the removal of plaque from the teeth during the residence time in the oral cavity. Different sizes of granules in the granular powders can increase or decrease the size of the small ridges or bumps and as such, alter the abrasive cleaning properties of the composition. As the oral tissues are cleansed, the encapsulation dissolves and releases Bio-available Calcium and Phosphate. This mechanism would not be known to a person having ordinary skill in the art that Bio-available ions could be administered in this method. Additionally, the texturing on the candy imparted by the combination of calcium-, phosphate-, and fluoride-containing compounds stimulates the salivary glands causing increased salivation. This increased salivation further causes increased oral movement, ensuring thorough coating of the tooth enamel with the calcium-, phosphate-, and fluoride-containing compounds.

In some embodiments, the composition and delivery vehicle may also comprise therapeutic pharmaceuticals, such as pain relievers, cough medicines, or antibiotics. These therapeutic pharmaceuticals would follow the timed-release behavior of the composition based on the residence time in the oral cavity. Any number of different pharmaceuticals would be known to a person having ordinary skill in the art and could be administered in this method.

It is to be understood that the invention is not limited to the exact details of construction, operation, exact materials or embodiments shown and described, as obvious modifications and equivalents will be apparent to one skilled in the art. While the specific embodiments have been illustrated and described, numerous modifications come to mind without significantly departing from the spirit of the invention, and the scope of protection is only limited by the scope of the accompanying Claims.

We claim:

1. A therapeutic composition for improving oral health comprising:
at least one remineralizing agent, comprising:
at least one fluoride-containing compound;
at least one calcium-containing compound;
at least one phosphate-containing compound;
calcium carbonate;
at least one sweetener;
an additive, selected from one or more of the following:
at least one tooth whitening agent;
at least one probiotic;
at least one energy supplement;
at least one hydrating agent; and
at least one treatment agent for halitosis,
at least one electrolyte,
at least one germicidal agent.

2. The therapeutic composition of claim 1, wherein the at least one calcium-containing compound is tricalcium phosphate.

3. The therapeutic composition of claim 1, wherein said at least one agent to treat halitosis, is a bacteria.

4. The therapeutic composition of claim 1, wherein the calcium carbonate is present in an amount between about 0.2 mg to about 20 mg.

5. The therapeutic composition of claim 1, further comprising caffeine.

6. The therapeutic composition of claim 1, wherein the at least one sweetener is selected from the group consisting of a high-intensity sweetener in a range of about 0.001 g to about 0.006 g, and an alcohol-containing sweetener in the range of about 1 g to about 6 g.

7. The therapeutic composition of claim 6, wherein said high-intensity sweetener is stevia and said alcohol containing sweetener is erythritol.

8. The therapeutic composition of claim 1, wherein the composition is in a form of soft candy.

9. The therapeutic composition of claim 8, wherein said form of soft candy is in a form selected from the group consisting of: a hard candy, a chewy candy, a gummy candy, and a jelly candy.

10. The therapeutic composition of claim 9, wherein said form of hard candy is a lollipop.

11. The therapeutic composition of claim 1, wherein the composition is in a form of a powder.

12. The therapeutic composition of claim 1, wherein the calcium carbonate is present in a quantity sufficient to buffer a consumer's oral cavity with a pH of about 7 or greater.

13. A method of improving oral health, the method comprising the steps of:
neutralizing the oral cavity pH by administering calcium carbonate;
maintaining a steady state of neutral pH in the oral cavity,
reducing the oral bacterial load, and
simultaneously remineralizing tooth enamel.

14. The method of improving oral health of claim 13, wherein the method further comprises administering a delivery vehicle comprising:
at least one remineralizing agent comprising:
at least one fluoride-containing compound;
at least one calcium-containing compound;
at least one phosphate-containing compound;
calcium carbonate;
at least one sweetener;
at least one additive comprising:
at least one tooth whitening agent;
at least one probiotic;
at least one hydrating agent;
at least one energy supplement,
at least one treatment agent for halitosis,
at least one electrolyte, and
at least one germicidal agent.

15. The method of improving oral health of claim 14, wherein the at least one calcium-containing compound further comprises tricalcium phosphate.

16. The method of improving oral health of claim 14, wherein the at least one sweetener is selected from the group consisting of a high-intensity sweetener in a range of about 0.001 g to about 0.006 g, and an alcohol-containing sweetener in the range of about 1 g to about 6 g.

17. The method of improving oral health of claim 14, wherein the at least one treatment agent for halitosis further comprises a bacteria.

18. The method of improving oral health of claim 14, wherein the at least one agent to combat halitosis further comprises lactobacillus.

19. The method of improving oral health of claim 14, wherein the calcium carbonate buffers a consumer's oral cavity to a pH of about 7 or greater.

20. The method of improving oral health of claim 14, wherein the buffer solution in the oral cavity of steady state pH comprises a pH of at least 6.9 or greater.
